(19) United States
(12) Patent Application Publication
(10) Pub. No.: US 2009/0069833 A1
(43) Pub. Date: Mar. 12, 2009

(54) DISPOSABLE LANCET

(76) Inventor: Fu-Yuan LI, Wan Tan Hsiang (TW)

Correspondence Address: LEONG C LEI, PMB # 1008, 1867 YGNACIO VALLEY ROAD, WALNUT CREEK, CA, 94598, US

(21) Appl. No.: 11/853,848

(22) Filed: Sep. 12, 2007

(51) Int. Cl. A61B 5/151

(52) U.S. Cl. 606/182

(57) ABSTRACT

The disposable lancet mainly contains a resilient needle body with two arms extended forward into a Y shape with a needle between the two arms. The two arms and the needle of the needle body are housed inside a sleeve member and the combination is slidably installed inside an enclosure. When the lancet is not yet used, the arms are blocked by two wedges of the sleeve member and the needle is hidden and protected inside the sleeve member. By compressing the sleeve member into the enclosure, the arms are forced out of the blockage and the needle body is driven forward by its resilience to penetrate through the sleeve member to draw blood. As the needle body retreats back into the sleeve member, the sleeve member is locked by the enclosure and cannot return to its un-used state.

### TECHNICAL FIELD OF THE INVENTION

The present invention generally relates to lancets, and more particularly to a lancet that can be used only once.

### DESCRIPTION OF THE PRIOR ART

Most conventional lancets are not wasted automatically after use and therefore there is a significant hazard for infection. Even though there are indeed some disposable lancets available in the industry, they usually have a complex structure, a difficult manufacturing process, and therefore a high production cost. The disposable lancets are as such not yet widely popular. In addition, most these lancets don't have protection means to guard against accidental injuries by the needle. Their use to senior citizens is also quite difficult.

### SUMMARY OF THE INVENTION

Accordingly, a novel disposable lancet is provided herein. The disposable lancet mainly contains a resilient needle body with two arms extended forward into a Y shape with a needle between the two arms. The two arms and the needle of the needle body are housed inside a sleeve member and the combination is slidably installed inside an enclosure.

When the lancet is not yet used, the arms are blocked by two wedges of the sleeve member and the needle is hidden and protected inside the sleeve member. By compressing the sleeve member into the enclosure, the arms are forced out of the blockage of the sleeve member and the needle body is driven forward by its resilience to penetrate through a front opening of the sleeve member to draw blood. As the needle body retreats back into the sleeve member, the sleeve member itself is locked by the hooks of the enclosure and cannot return to its un-used state. The lancet therefore can only be used once.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective sectional view showing the inside of a disposable lancet according to an embodiment of the present invention.

FIG. 2 is a perspective exploded view showing the various components of the disposable lancet of FIG. 1.

FIG. 3 is a perspective sectional view showing the inside of the sleeve member of FIG. 1.

FIG. 4 is a perspective sectional view showing the inside of the enclosure of FIG. 1.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

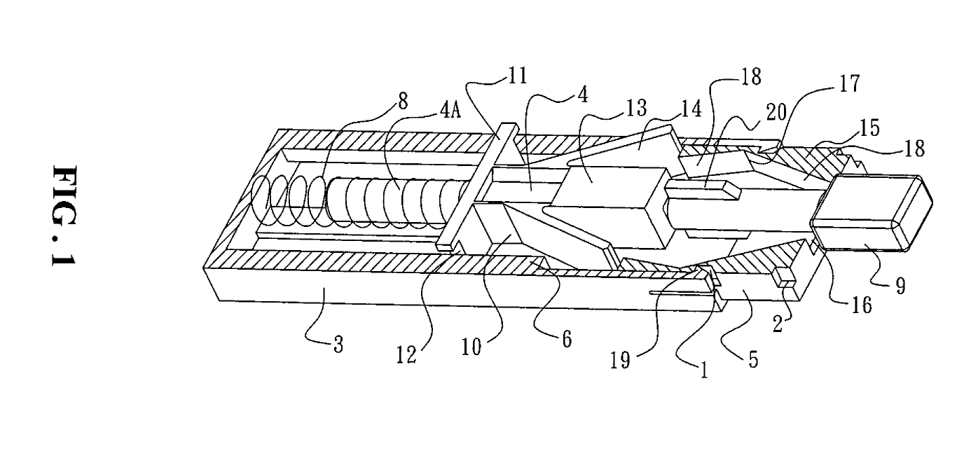

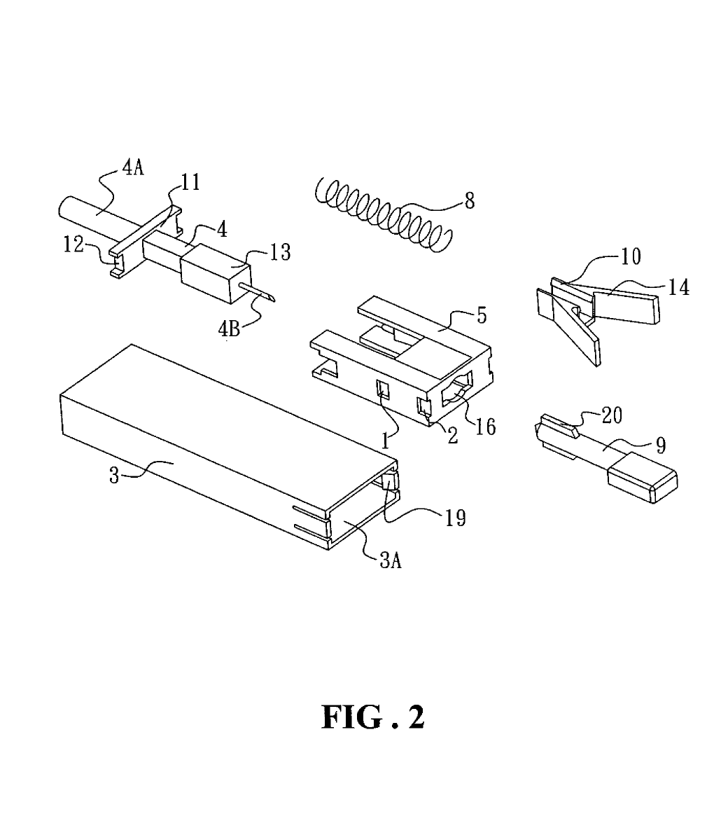

As shown in FIGS. 1 and 2, a disposable lancet according to an embodiment of the present invention has an elongated needle body (not numbered) which contains a cylindrical end segment 4A and a rectangular middle segment 4 with one of their ends attached to two major surfaces of a rectangular blocking plate 11, respectively. The other end of the middle segment 4 is attached to one end of a rectangular end segment 13. A needle 4B is extended from the other end of the end segment 13. The segments 4A, 4, 13 and the needle 4B are axially aligned. Each of the two shorter sides of the rectangular blocking plate 11 has a notch 12. The middle segment 4 has a smaller cross section than that of the end segment 13. In the following, the direction the needle 4B is pointing is referred to the front and therefore the opposite direction is the back. The shorter sides of the blocking plate 11 are said to be at the left and right, respectively, and the longer sides of the blocking plate are said to be at the top and bottom.

The disposable lancet further contains a Y-shaped member 10 made of a plastic material. The member 10 has a U-shaped body open to the top and, from the member 10's left and right sides, two arms 14 are extended slantwise towards the front. The needle body and the member 10 are joined together by embedding the middle segment 4 inside the U-shaped body of the member 10. The end segment 13 and the needle 4B are thereby positioned right between the two arms 14. The combination of the needle body and the member 10 is then plugged into a back side of a hollow and rectangular sleeve member 5.

The sleeve member 5 has an opening 16 at its front side. The opening 16 has two purposes. On one hand, it allows a tubular cover 9 to thread through so that the needle 4B is received and protected inside a back end of the tubular cover 9. On the other hand, after the tubular cover 9 is removed, the opening 16 allows the needle 4B to run through for drawing blood. Please note that two ridges 20 correspondingly on the top and bottom of the back end, respectively, are extended from the back end of the tubular cover 9 towards the front for an appropriate distance.

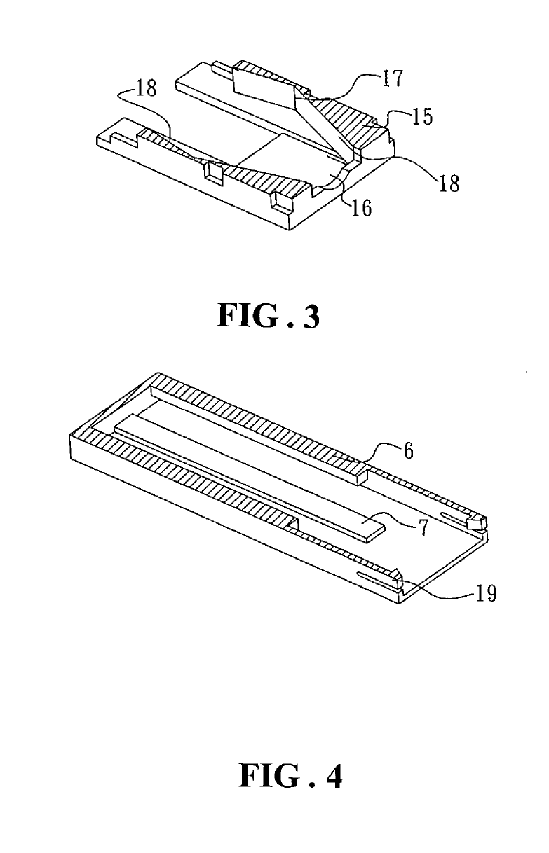

As shown in FIG. 3, along the left inner wall of the sleeve member 5, there is a first wedge 18 extending from adjacent to the back side and a second wedge 15 extending from the front side of the sleeve member 5, slanting towards each other and towards the left side of the sleeve member 5. The first and second wedges 18 and 15 therefore interface with each other at a junction 17. Another first wedge 18 and another second wedge 15 are provided identically and correspondingly along the right side of the sleeve member 5. As such, when the combination of the needle body and the member 10 is plugged into the back side of the sleeve member 5, the two arms 14 are blocked by the back ends of the wedges 18.

Then, a resilient element 8 such as a helix spring is sleeved over the end segment 4A of needle body and is engaged by the blocking plate 11. The combination of the resilient element 8, the needle body, the Y-shaped member 10, and the sleeve member 5 are plugged into a rectangular enclosure 3 via its front opening 3A. The enclosure 3 is sealed at all other sides.

As shown in FIG. 4, there are two rails 6 correspondingly provided along the left and right inner walls of the enclosure 3, respectively. The rails 6 match the notches 12 of the needle body so as to guide the needle body in its front-back advancement when the needle body is pressed towards the back (thereby compressing the resilient element 8) or when the needle body is propelled to the front (as the resilient element 8 expands). Please note that the rails 6 extend from the back side to an appropriate location in the middle of the enclosure 3.

Along the left and right outer walls of the sleeve member 5, there are first notches 1 in the middle and second notches 2 right besides the front side of the sleeve member 5, respectively. On the other hand, the enclosure 3 has two flexible hooks 19 along the left and right inner walls right besides the front opening 3A, respectively. As such, when the combination of the resilient element 8, the needle body, the Y-shaped member 19, and the sleeve member 5 are plugged into the enclosure 3, the flexible hooks 19 are locked to the first notches 1, respectively.

Along the bottom inner wall of the enclosure 3, a flat rectangular piece 7 is extended from the back end to an appropriate location in the middle of the enclosure 3. The purpose of the piece 7 is to prevent the tubular cover 9 from compressing the sleeve member 5 further into the enclosure 3 by accident.

The operation of the disposable lancet is described as follows. First, the tubular cover 9 is removed and the enclosure 3 is vertically held so that the opening 16 of the sleeve member 5 is attached directly to the spot where the blood is to be drawn. Then, the enclosure 3 is slid downward and the sleeve member 5 is thereby forced into the enclosure 3. In the process, the resilient element 8 is compressed until the arms 14 of the Y-shaped member 10 are forced out of the blockage of the first wedges 18 by the front ends of the rails 6. When that happens, the compressed resilient element 8 suddenly expands and drives the needle body along with the Y-shaped member 10 and the needle 4B forward. The needle 4B therefore penetrates through the opening 16 into a user's skin to draw blood. Then, as the resilient element 8 restores itself, the needle body along with the Y-shaped member 10 and the needle 4B retreat into the enclosure 3. The front ends of the arms 14 will be again blocked at the junctions 17 and the hooks 19 will lock to the second notches 2. In this way, the sleeve member 5 and the needle body cannot return to their original, un-used state. The disposable lancet therefore cannot be re-used. Please note that the advancement of the needle body in the foregoing penetration and retreat process is at all times guided by the rails 6 for a smooth and reliable blood-drawing operation.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A disposable lancet, comprising:
a needle body having a first end segment, a middle segment, a blocking plate whose two major surfaces attach an end of said first end and middle segments respectively, a second end segment having an end connecting to the other end of said middle segment, and a needle extending from the other end of said second end segment pointing to a front direction, wherein said middle segment has a smaller cross section than that of said first end segment and said first end segment, middle segment, second end segment, and needle are aligned;
a resilient element positioned at a back end of said needle body and engaged by said needle body;
a Y-shaped member having a body and two arms extending from a left side and a right side of said body toward said front direction wherein said body is joined with said middle segment so that said second end segment and said needle are between said arms;
a sleeve member having a front opening and receiving at least said Y-shaped member, said second end segment and said needle from a back side, wherein said arms are blocked by two wedges provided along left and right inner walls of said sleeve member; and
a hollow enclosure having a front opening and receiving said resilient element, said needle body, said Y-shaped member, and said sleeve member from said front opening of said enclosure wherein said enclosure has at least a rail extending from a back end to an appropriate location in the middle along a lateral inner wall of said enclosure;
wherein, when said sleeve member is compressed towards a back direction into said enclosure, said resilient element is compressed until when said arms break out of blockage by said rail; and said resilient element expands to drive said needle body, said Y-shaped member, and said needle towards said front direction and said needle thereby penetrates through said front opening of said sleeve member.

2. The disposable lancet according to claim 1, further comprising a cover removably plugging into said front opening of said sleeve member and having a back end receiving said needle.

3. The disposable lancet according to claim 2, wherein said enclosure has at least an elongated piece at an inner bottom side extending from said back end of said enclosure to an appropriate location in the middle; said cover has at least a ridge at said back end of said cover; and said ridge of said cover is blocked by said piece.

4. The disposable lancet according to claim 1, wherein said blocking plate has at least a notch at a lateral side of said blocking plate; and said notch matches said rail so that said needle body's advancement is under the guidance of said rail.

5. The disposable lancet according to claim 1, wherein said enclosure has at least a hook right besides said front opening of said enclosure; said sleeve member has at least a first notch and a second notch along a lateral side of said sleeve member; said first notch is in the middle of said lateral side; said second notch is right besides said front opening of said sleeve member; when said sleeve member is not compressed, said hook locks said first notch; and, when said sleeve member is compressed into said enclosure, said hook locks said second notch of said sleeve member.
